(19) United States
(12) Patent Application Publication
(10) Pub. No.: US 2012/0260825 A1
(43) Pub. Date: Oct. 18, 2012

(54) POLYMERIC DISPERSANTS

(76) Inventors: Tatiana N. ROMANOVA, Loveland, OH (US); Russell J. SCHWARTZ, Cincinnati, OH (US)

(21) Appl. No.: 13/528,356

(22) Filed: Jun. 20, 2012

Related U.S. Application Data
(62) Division of application No. 12/301,743, filed on Nov. 20, 2008.
(60) Provisional application No. 60/803,217, filed on May 25, 2006.

(51) Int. Cl. C07C 231/02; C07D 487/04; C07C 327/30; C08K 5/42; C08K 5/20; C08K 5/372; C08K 5/3417; C08K 5/3462; C07D 209/48; C07D 295/13

(52) U.S. Cl. 106/505; 562/442; 548/477; 562/451; 548/433; 560/37; 558/256; 544/400; 544/372; 544/357; 106/506; 524/94; 524/217; 524/89; 524/100

(57) ABSTRACT

Dispersants for pigments as well as dispersions containing them, and compositions such as inks and coatings containing them are described. The dispersants are the reaction product of at least one dianhydride with at least two different reactants, each of which can be an amine, alcohol, or thiol, and at least one of which is polymeric.

### BACKGROUND OF THE INVENTION

This invention relates to polymeric pigment dispersants useful for dispersing a wide variety of pigments.

Pigments are dispersed as fine particles into a liquid medium for use in inks (including ink jet formulations) and coatings, as well as plastics, cosmetics, adhesives, and the like. It is desirable for the pigment to be dispersed as finely as possible and as rapidly as possible into the liquid medium and remain as a stable fine dispersion over time for optimum results. Unfortunately, the dispersion of fine particles in liquids is unstable in that the particles tend to agglomerate or flocculate causing uneven pigmentation, changes in rheology, and changes in color value over time in the product where pigmentation is desired.

To minimize the effects of agglomeration and/or flocculation, numerous pigment dispersants have been developed. They have been used to form disperse pigments in a variety of water borne and solvent borne coating compositions. Examples of U.S. patents describing dispersants and pigment dispersants include U.S. Pat. Nos. 4,496,686, 4,673,705, 4,754,056, 5,034,444, 5,424,364, 6,037,414, 6,451,950, 6,495,618, and 6,878,799.

The dispersants act to lower the viscosity of pigment dispersions. As a result, more pigment can be employed, affording very concentrated dispersions which can be satisfactorily handled and dispersed. However, the upper limit of pigment concentration in concentrated pigment dispersions is determined by the viscosity, and as the pigment concentration increases linearly, the viscosity of the dispersion increases exponentially. When the viscosity becomes too high, a pigment can no longer be dispersed satisfactorily or handled during ink or coating manufacturing. In addition, the system experiences excessive temperature increases due to the frictional forces that occur during the dispersion process. This can cause degradation of certain pigments and inks or coating resins. Incorporation of the appropriate dispersant helps to eliminate or minimize such defects by providing a more efficient dispersion and dispersion stabilization.

The present invention provides new polymeric dispersants.

### SUMMARY OF THE INVENTION

The present invention relates to dispersants for pigments and to dispersions containing them, as well as compositions such as inks (including ink jet), coatings or plastics containing them and articles made from substrates treated with inks and coatings or with plastics containing these dispersants. The new dispersants provide reduced viscosity for the pigment dispersions which can be solvent based, water based, energy curable or a combination of such systems. Inks, coatings and plastics containing the surface modified pigments or the dispersions are also provided as are substrates decorated by these inks and coatings and articles made from substrates or plastics colored by the dispersed pigments.

The dispersants of the present invention are the reaction product of at least one dianhydride with at least two different reactants, where at least one reactant is polymeric, and each of which reactants contain primary or secondary amine, hydroxyl, or thiol functionality. Such products may be further modified by cyclization, salt formation, or other functional group modification.

### BRIEF DESCRIPTION OF THE DRAWINGS

FIG. 1 shows the reaction scheme of example 1.

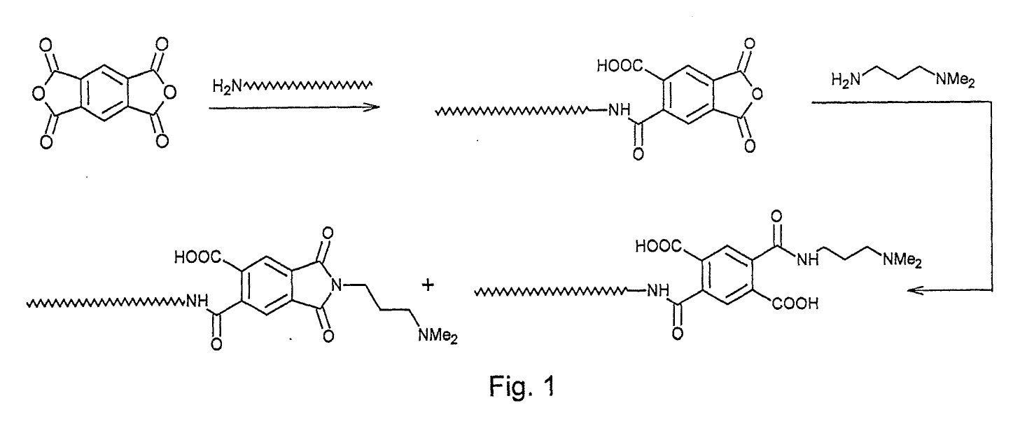

FIG. 2 shows the reaction scheme of example 2.

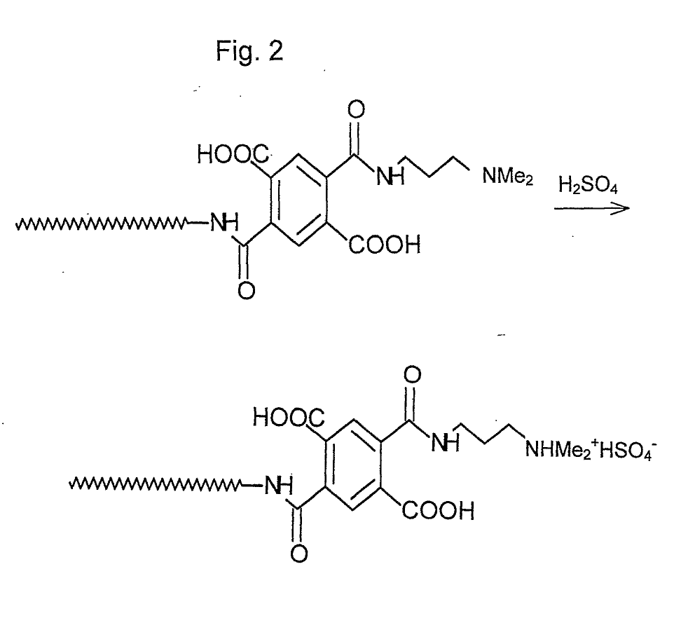

FIG. 3 shows the reaction scheme of example 3.

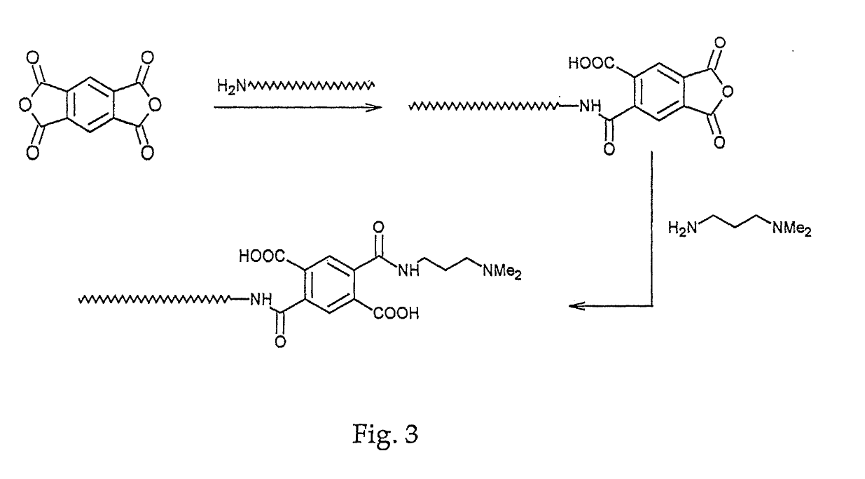

FIG. 4 shows the reaction scheme of example 4.

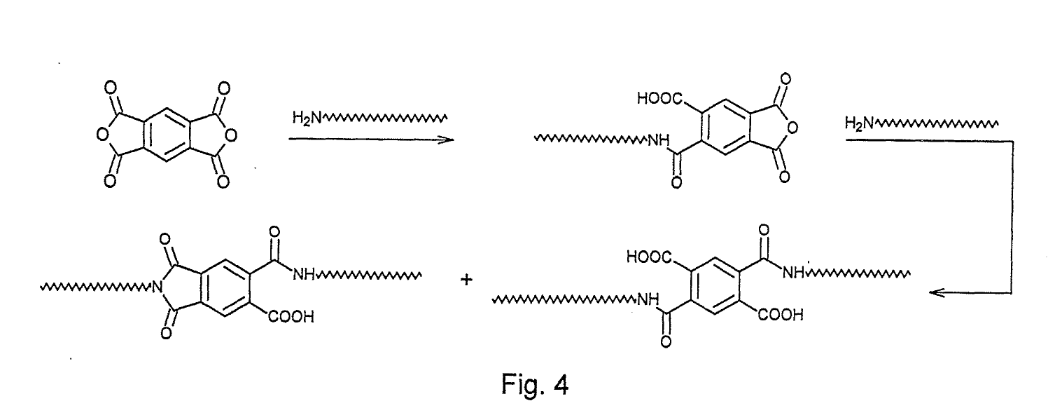

FIG. 5 shows the reaction scheme of example 5.

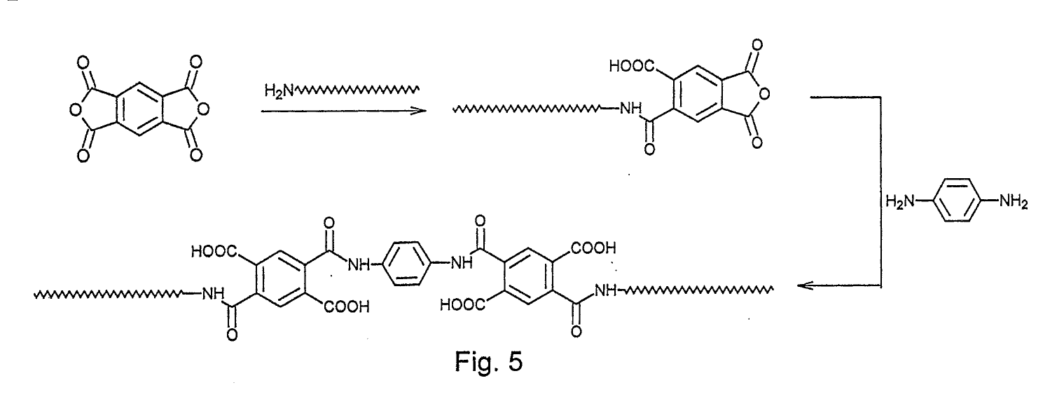

FIG. 6 shows the reaction scheme of example 6.

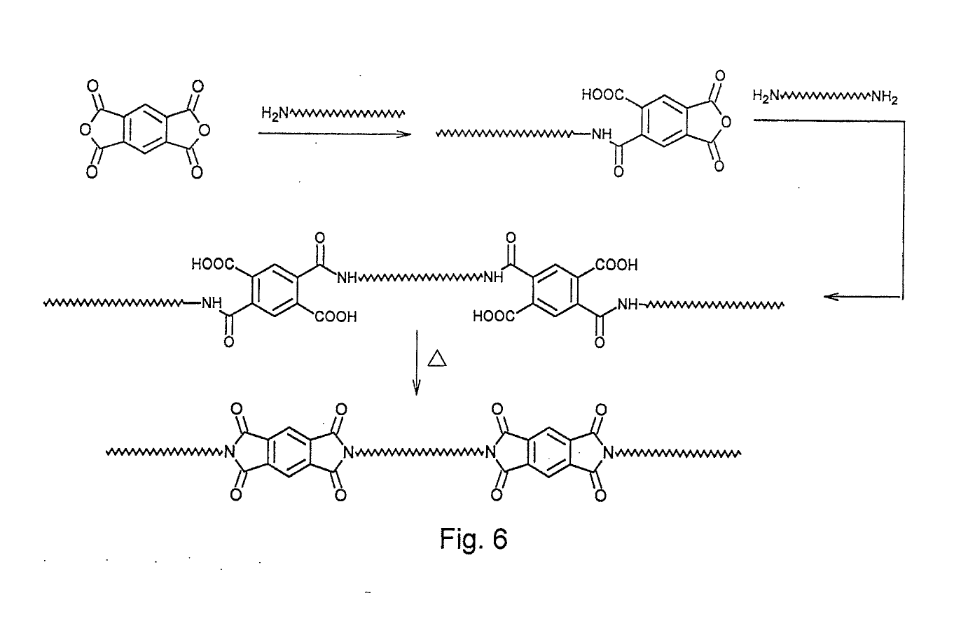

FIG. 7 shows the reaction scheme of example 7.

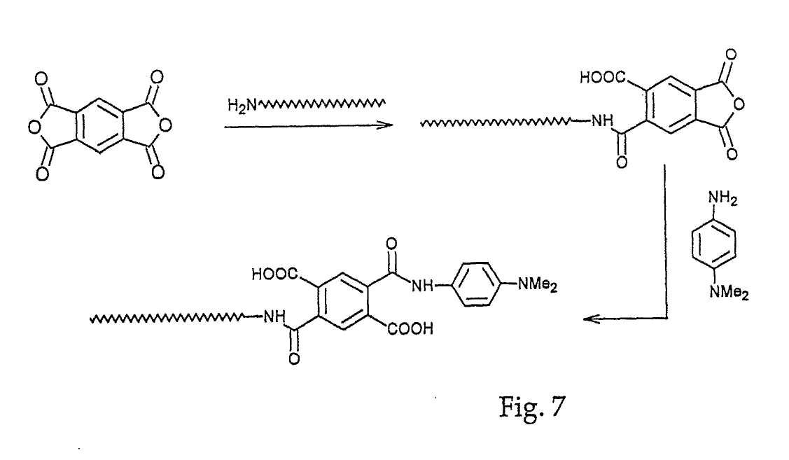

FIG. 8 shows the reaction scheme of example 8.

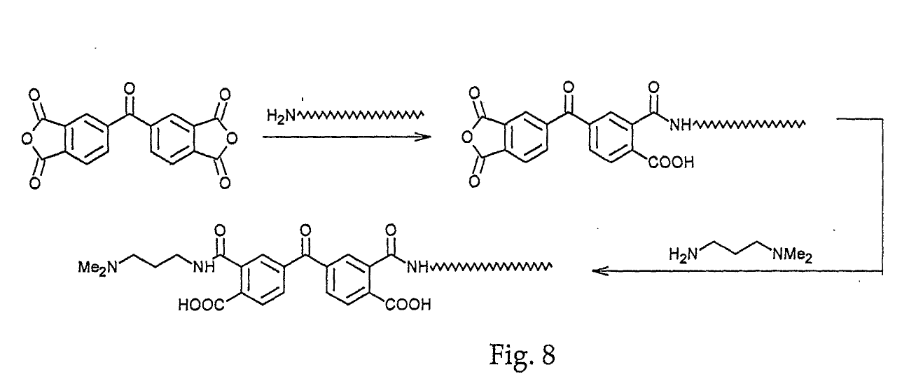

FIG. 9 shows the reaction scheme of example 13.

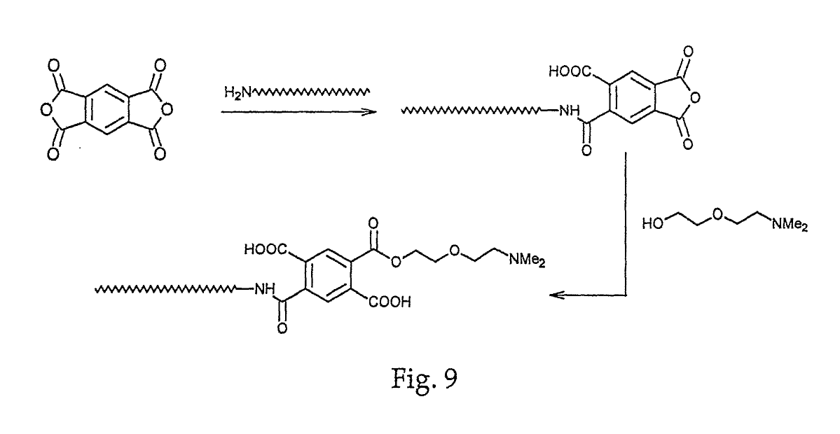

FIG. 10 shows the reaction scheme of example 14.

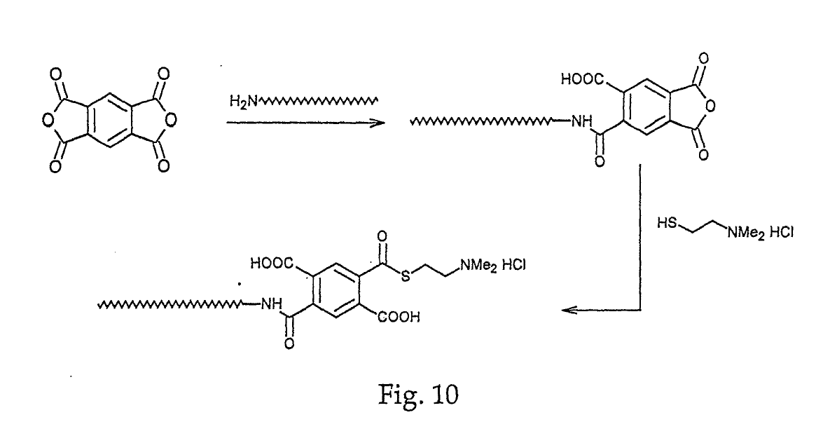

FIG. 11 shows the reaction scheme of example 15.

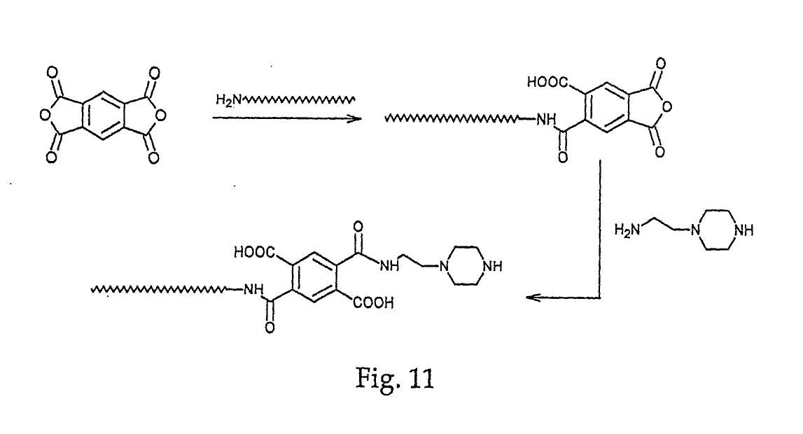

FIG. 12 shows the reaction scheme of example 16.

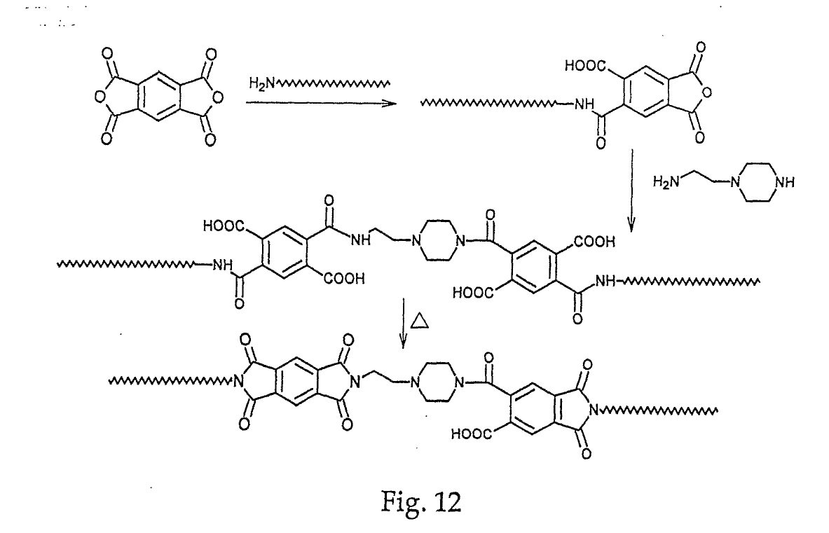

FIG. 13 shows the reaction scheme of example 17.

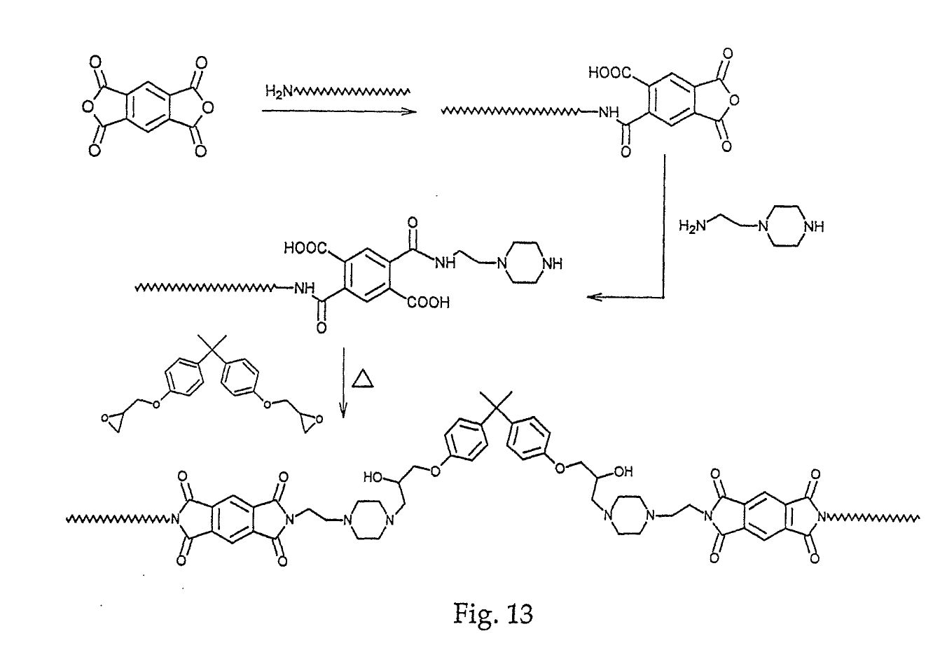

FIG. 14 shows the reaction scheme of example 18.

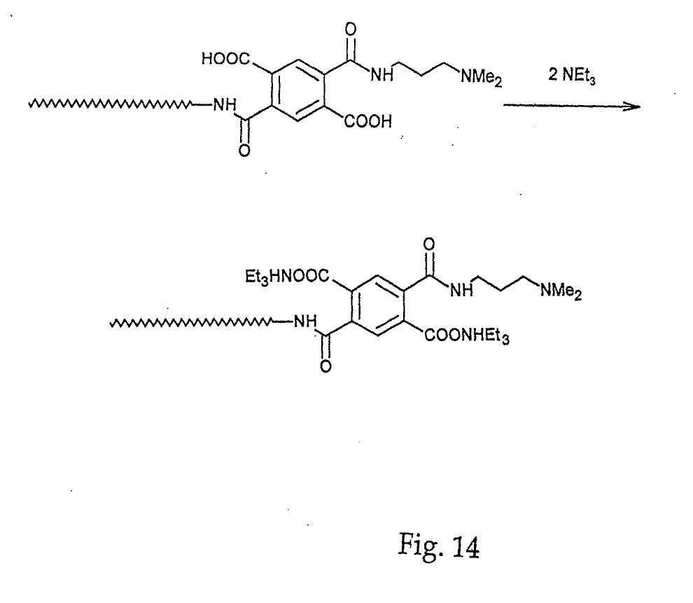

In all of the drawings, the wavy line is intended to indicate the polymeric nature of the moiety and not to indicate any particular number of atoms, functionalities, substituents or structures.

### DESCRIPTION OF THE INVENTION

In accordance with the present invention, new pigment dispersants, surface modified pigments or pigment dispersions, and ink (including ink jet) and coating containing them are provided, as are substrates decorated by these inks and coatings, and articles made from these substrates including plastics colored with the dispersed pigments or articles made from the colored plastics.

The novel pigment dispersants of the present invention are particularly effective for the dispersion of both inorganic pigments and organic pigments. Any known pigments can be used to form pigment dispersions using these dispersants. Examples of suitable pigments include metallic oxides such as titanium dioxide, iron oxides of various colors, and zinc oxide; carbon black; filler pigments such as talc, china clay, barytes, carbonates, and silicates; a wide variety of organic pigments such as quinacridones, diketopyrrolopyrroles, phthalocyanines, perylenes, azo pigment, and indanthrones carbazoles such as carbazole violet, isoindolinones, isoindolones, isoindolines, thioindigio reds, benzimidazolones and benzimidazolinones; and metallic flakes such as aluminum flake, pearlescent flakes, and the like.

The pigments suitable for use in the present invention thus include International Colour Index or C.I. Pigment Black 1, C.I Pigment Black 31, C.I. Pigment Black 11, C.I. Pigment Black 7, C.I Pigment Black 250, C.I. Pigment Blue 61, C.I. Pigment Blue 62, C.I. Pigment Blue 15, C.I. Pigment Blue 15:1, C.I. Pigment Blue 15:2, C.I. Pigment Blue 15:3, C.I. Pigment Blue 15:4, C.I. Pigment Blue 15:6, C.I. Pigment Blue 16, C.I. Pigment Blue 29, C.I. Pigment Blue 27, C.I. Pigment Green 17, C.I. Pigment Green 18, C.I. Pigment Green 7, C.I. Pigment Green 36, C.I. Pigment Orange 5, C.I. Pigment Orange 13, C.I. Pigment Orange 16, C.I. Pigment Orange 34, C.I. Pigment Orange 36, C.I. Pigment Violet 3, C.I. Pigment Violet 27, C.I. Pigment Violet 23, C.I. Pigment Violet 19, C.I. Pigment Red 122, C.I. Pigment Red 202, C.I. Pigment Red 206, C.I. Pigment Red 207, Pigment Red 254, C.I. Pigment Red 22, C.I. Pigment Red 23, C.I. Pigment Red 17, C.I. Pigment Red 210, C.I. Pigment Red 170, C.I. Pigment Red 188, C.I. Pigment Red 185, C.I. Pigment Red 146, C.I. Pigment Red 144, C.I. Pigment Red 101, C.I. Pigment Red 176, C.I. Pigment Red 48:1, C.I. Pigment Red 48:2, C.I. Pigment Red 57:1, C.I. Pigment Red 81:1, C.I. Pigment Red 81:2, C.I. Pigment Red 81:3, C.I. Pigment Red 81:5, C.I. Pigment Red 179, C.I. Pigment Red 3, C.I. Pigment Red 249, C.I. Pigment Red 114, C.I. Pigment Red 181, C.I. Pigment Yellow 1, C.I. Pigment Yellow 2, C.I. Pigment Yellow 3, C.I. Pigment Yellow 147, C.I. Pigment Yellow 142, C.I. Pigment Yellow 42, C.I. Pigment Yellow 151, C.I. Pigment Yellow 154, C.I. Pigment Yellow 180, C.I. Pigment Yellow 138, C.I. Pigment Yellow 139, C.I. Pigment Yellow 93, C.I. Pigment Yellow 109, C.I. Pigment Yellow 110, C.I. Pigment Yellow 14, C.I. Pigment Yellow 12, C.I. Pigment Yellow 17, C.I. Pigment Yellow 13, C.I. Pigment Yellow 74, C.I. Pigment Yellow 73, C.I. Pigment Yellow 75, C.I. Pigment Yellow 83, C.I. Pigment Yellow 65, C.I. Pigments Yellow 128, D&C Red No. 7, D&C Red No. 6 and D&C Red No. 34. Other pigment examples include carbon black pigments such as Regal 330 (available from Cabot Corporation); quinacridone pigments such as Quinacridone Magenta (228-0122) (available from Sun Chemical Corporation); diarylide yellow pigments such as AAOT Yellow (274-1788) (available from Sun Chemical Corporation); and phthalocyanine blue pigments such as Blue 15:3 (294-1298) (available from Sun Chemical Corporation). Usable pigments are not limited to the foregoing.

The dispersants of the present invention are the reaction product of at least one dianhydride with at least two reactants, each of which can be a primary or secondary amine, alcohol or thiol, and at least one of which is polymeric.

The tetracarboxylic dianhydrides which are useful in the present invention may be represented by the formula:

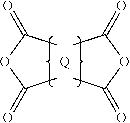

in which Q is a carbon-containing linking group which may be linear, branched or cyclic aliphatic or aromatic group, or combination thereof, and can be saturated or unsaturated (isolated or conjugated), and can contain H, O, N, S, P, Si and/or halogen atoms in addition to carbon. Q can thus contain substructures such as alkyl, alkenyl, alkynyl, alkylene, alkenylene, alkynylene, aryl, arylene or combinations thereof, as well as such groups containing O, N, S, P, Si and/or halogen in their main chain or in a side chain. Illustrative examples include pyromellitic dianhydride, biphenyltetracarboxylic dianhydride, benzenophenone-tetracarboxylic dianhydride, naphthalenetetracarboxylic dianhydride, tetrahydro-3,3′-dimethyl-[3,3′-bifuran]-2,2′5,5′-tetrone; 3,3′-(1,2-ethanediyl)-bis[dihydro-2,5-furandione] and the like. Additional dianhydrides include but not limited to cyclobutane-1,2,3,4-tetracarboxylic dianhydride, ethylenediaminetetraacetic dianhydride, tetrahydrofurantetracarboxylic dianhydride, bicyclo[2.2.2]oct-7-ene-2,3,5,6-tetracarboxylic dianhydride. Of the foregoing compounds, pyromellitic dianhydride is preferred.

The dianhydride is reacted with two or more different reactants, which can contain primary or secondary amine, hydroxyl or thiol functionality, at a temperature of about 0° to 200° C., preferably at about 20° to 45° C., whereby each anhydride can assymmetrically react with these reactants. Individual reactants may contain one or more than one amino, hydroxyl and/or thiol group, or a combination thereof.

Each of the reactants generally conforms to the formula (XH)ₘ—R—(YH)ₙ in which X and Y are independently NZ, O or S, m and n are independently 0, 1 or 2, m plus n is at least 1, and R and Z are independently hydrogen or linear, branched or cyclic aliphatic or aromatic group, or combination thereof, which may be saturated or unsaturated (isolated or conjugated), such as alkyl, alkylene, alkenyl, alkenylene, alkynyl, alkynylene, aryl, arylene, as well as such groups containing O, N, S, P, Si and/or halogen in their main chain or in a side chain, or combinations thereof, provided that at least one reactant is polymeric. Polymeric reactants are those containing a polymeric group comprising the same repeating monomer units (homopolymer) or multiple monomer units (copolymer), or both, where the monomer can be any type of monomer. Such copolymers can be further classified as random, alternating, graft, block, branched, comb-like polymers, or combinations thereof.

In some cases R and Z can form heterocyclic ring with N. Such examples of reactants include, but are not limited to, piperazine, 1-(2-aminoethyl)piperazine, 1-(2-hydroxyethyl)piperazine and 1-piperazinepropanol.

The R and/or Z groups can be unsubstituted or substituted by, for instance, one or more functional groups. Examples of functional groups include, but are not limited to, hydroxyl, carboxyl, halogen, cyano, primary, secondary or tertiary amino, thiol, sulfonate, sulfates, phosphate, phosphonate, and the like.

Examples of reactants include, but are not limited to, water, hydroxylamine, alcohols such as methanol, ethanol, propanol, isopropanol, butanol, hexanol, ethylene glycol, propylene glycol, dimethylaminoethanol, diethylamino ethanol, dimethylaminopropanol, diethylaminopropanol, (dimethylamino)benzyl alcohol, (dimethylamino)-phenylethanol, 2-[2-(dimethylamino)ethoxy]ethanol and the like; amines such as NH₃, methylamine, dimethylamine, ethylamine, n-propylamine, n-butylamine, n-hexylamine, β-hydroxyethylamine, hydroxylamine, hydrazine, methylhydrazine, 1,1-dimethylhydrazine, 1,2-dimethylhydrazine, phenylhydrazine, 1-methyl-1-phenylhydrazine, 1-acetyl-1-phenylhydrazine, dimethylaminoethylamine, diethylaminoethylamine, 2-ethylhexylaminoethylamine, stearylaminoethylamine, oleylaminoethylamine, dimethylaminopropylamine, dibutylaminopropylamine, diethylaminobutylamine, dimethylaminoamylamine, diethylaminohexylamine, piperidinomethylamine, piperidinoethylamine, piperidinopropylamine, 1-(2-aminoethyl)piperazine, 1,4-bis (3-aminopropyl)piperazine, 1-piperazinepropanol, 1-(2-hydroxyethyl)piperazine, N-(3-aminopropyl)-2-pipecoline, 1-(3-aminopropyl)imidazole, 1-(4-aminophenyl)-1H-imidazole, morpholinoethylamine, morpholinopropylamine, 1,2-cyclopentanediamine, 1,2-cyclohexanediamine, o-phenylenediamine, 2,3- or 1,8-diaminonaphthalene, 2,3- or 3,4-diaminopyridine, 9,10-diaminophenanthrene, N,N-dimethyl-1,4-phenylenediamine, and the like; and thiols such as methanethiol, ethanethiol, methanethiol, propenethiol, butanethiol, hexanethiol, decanethiol, dodecanethiol, 2-(dimethylamino)ethanthiol, (3-nitrobenzyl)mercaptan, benzyl mercaptan, and the like.

The polymeric reactant has one or two primary or secondary amine-, hydroxyl- or thio end groups or combinations thereof. The poly(alkylene oxide) amines in which the alkylene oxide group contains 1 to about 5 carbon atoms. Those containing 2 or 3 carbon atoms are preferred and are well known, commercially available materials. These amines contain a polyether backbone that is based either on propylene oxide, ethylene oxide or mixed propylene oxide and ethylene oxide. The alcohols which were aminated to form the amines, as well as the corresponding thiols, can also be employed. For the purposes of the present invention, the poly(oxyalkylene)amines are preferred.

At least one such polymeric reactant is employed. All of the reactants can be polymeric provided that at least two different reactants are employed. It is the function of a difunctional reactant to link two or more dianhydride compounds together. Difunctional reactants can be symmetric or asymmetric, and have primary or secondary amino, hydroxyl or thio functional groups or combinations thereof.

In general, mono-functional polymeric reactants that are useful in this invention have equivalent molecular weight between about 200 to 5000, preferably between about 500 to 3000, and more preferably between about 1000 to 2000. Useful difunctional polymeric reactants have equivalent molecular weights from about 30 to 2500, preferably from about 50 to 1000, and more preferably from about 100 to 500.

The reaction is preferably conducted stepwise by slow addition of the first reactant to a solution of dianhydride or mixture of dianhydrides in order to facilitate addition of one reactant to one side of the dianhydride while leaving the other anhydride substantially intact, followed by addition of the other reactant(s) to the second anhydride group. It is preferable to conduct the reaction in a substantially anhydrous and inert solvent system in which the anhydride is stable.

Any solvents in which the dianhydride is dissolved and is stable can be used, such as ketones and esters with relatively low boiling point if their subsequent removal is required or desired. Solvents can also be selected to remain in the composition if they are compatible in the end application. The preferred solvents are acetone and 2-butanone. The system need not be completely anhydrous in that a small quantity of water which does not substantially hydrolyze the anhydride group to a diacid can be employed.

The preferred dispersant produced contain a unit or units represented by the formulae

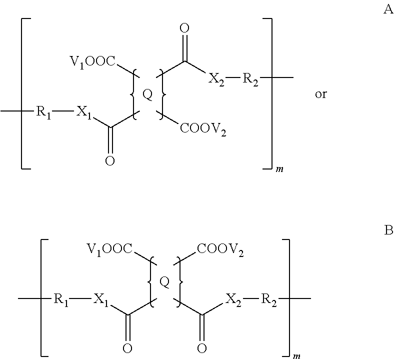

or both, in which Q is as set forth above, X₁ and X₂ are NZ, O or S, and m is an integer from 1 to about 10. X₁ or X₂ is preferably NZ. Each V₁ and V₂ independently are hydrogen or a residue of an entity reactive with COOH, such as organic or inorganic cation. Each R₁, R₂ and/or Z independently are hydrogen or linear, branched or cyclic aliphatic or aromatic groups, as well as such groups containing O, N, S, P, Si, halogen and/or metal ion in their main chain or in a side chain, and can be saturated or unsaturated (isolated or conjugated), such as alkylene, alkenylene, arylene, heteroarylene, heterocyclic groups and the like, or combinations thereof, which may contain ether, ester, carbonate, ketone, amino, amide, urea, urethane, C≡N and/or C═P moieties, or combinations thereof, provided that at least one R₁, R₂ or Z is polymeric. Polymeric materials are those containing a polymeric group comprising the same repeating monomer units (homopolymer) or multiple monomer units (copolymer), or both, where the monomer can be any type of monomer. Such copolymers can be further classified as random, alternating, graft, branched, block, and comb-like or combination thereof. The Q, R₁, R₂ and/or Z groups can be unsubstituted or substituted with one or more functional groups, which can be characterized as containing other atoms in addition to carbon and hydrogen. Each of the terminal groups of the dispersant will depend on the reactant(s) employed and can be independently hydrogen, halogen and/or any monovalent group corresponding to R. The presence of a function group in Q, R₁, R₂ and/or Z provides the ability to couple other entities to the molecule, as noted below.

When m is more than 1, two or more dianhydride moieties are connected by one or more difunctional reactants. When Z is H, one or more of the ring opened groups may become cyclized to form imides as a consequence of the time and temperature conditions employed. Such dispersants can have a unit or units represented by, but not limited to, the formulae:

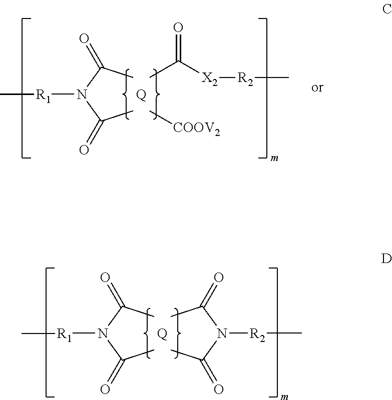

or any combination of cyclized or uncyclized units A through D thereof as part of the same molecule or a mixture of molecules.

Also, any functional group of the dispersant may be modified to form a dispersant with modified functional group. For example, a dispersant containing a tertiary amine group can be converted into salt (ammonium) form by treatment with an acid. The anion may be inorganic, such as halide, sulfate, phosphate, and the like, or organic, such as acetate, and the like. Any carboxylic or other acid groups of the dispersant can be converted into salt form by treatment with organic or inorganic base. Any functional group can undergo further modification. For example, a dispersant containing a secondary amino, hydroxyl or carboxyl group can be reacted with epoxy or isocyanate groups and the like. The specific modification is effected after the reaction of the dianhydride and reactants.

To form surface treated pigments an individual pigment or a mixture of pigments is predispersed with one or more dispersants of this invention in the customary solvent or solvent blend using conventional techniques such as high speed mixing, ball milling, sand grinding, attritor guiding, or two or three roll milling with further removal of solvent and the like.

To form a pigment dispersion or a millbase, an individual pigment or a mixture of pigments, which may or may not be surface treated, is combined with one or more dispersants of this invention in the customary solvent or solvent blend and dispersed using conventional techniques such as high speed mixing, ball milling, sand grinding, attritor guiding, or two or three roll milling, and the like. The pigment dispersion can also be made by grinding or milling the dry pigments with the dispersants of the present invention or by mixing the pigments with the dispersant in a liquid medium in a pigment flushing process. The resulting pigment dispersion, whether as a millbase or ink/coating will have a dispersant to pigment weight ratio of about 0.1/100 to 2/1, more preferably about 1/100 to 1/2, and most preferably about 1/20 to 1/5.

It may be desirable to add other optional ingredients to the pigment dispersion such as antioxidants, flow control agents, UV stabilizers, light quenchers and absorbers, and rheology control agents such as fumed silica and microgels. Film forming polymers such as acrylics, acrylourethanes, polyester urethanes, polyesters, alkyds, polyethers and the like, can also be employed.

The dispersant composition may further comprise a thermoset or a thermoplastic resin, crosslinking agents, flow and leveling agents, wetting agents, thickeners, anti-settling agents, UV stabilizers and other additives as required or desired to enhance the end use performance. The dispersants in this invention may be used together with other dispersants and/or dispersion aids, such as a grinding resin or an additive to achieve synergistic effects.

Other additives may be used in combination with the dispersant and pigment of the present invention. For instance, ammonia or some other suitably functional amine may be used to adjust the pH of the dispersion. Biocides, such as Proxel GMX (available from Rohm & Haas, Philadelphia, Pa.) can be used to aid in the inhibition of bacterial growth. Glycols may be used to modify the properties of the dispersion and improve jettability in the case of an ink jet ink, and would preferably include propylene glycol, polyethylene glycol (such as PEG 200) 400 and 1000, available from Union Carbide, Danbury, Conn.). Defoamers, co-solvents and surface active agents, such as octylphenolethoxylates or acetylenics, may be used to modify surface tension. However, the amount of additive should be held to a minimum.

The polymeric dispersant as such or as a millbase or as surface modified pigment is added to the ink or coating composition which may be a solvent based, water based or energy curable (ultra-violet, electron beam or cationic), such as monomers, oligomers, etc. or a combination of those systems. Examples of suitable solvents include but are not limited to aliphatic, cyclic and aromatic hydrocarbons, alcohols, esters, ketones, ethers, halogenated solvents or combination thereof. A pigment and dispersant, surface modified pigment or a preformed pigment dispersion can thus be added to a variety of aqueous or solvent borne inks or coatings. These compositions may contain film-forming polymers such as hydroxy functional acrylic and polyester resins and crosslinking agents such as blocked isocyanates, alkylated melamines, polyisocyanates, epoxy resins, and the like. Such inks and coatings can contain any known additive(s).

When formulated into an ink or coating, the pigment generally comprises about 0.1% up to about 70% by weight of the composition, preferably about 1% up to about 20%, and most preferably up to about 10%. The dispersant(s) will generally constitute about 0.1 to 50%, preferably less than 20% and more preferably less than 10% of the composition.

When a pigmented composition containing a dispersant of the present invention is heated above about 70° C., the mobility of the pigment in the composition may be reduced. This property is particularly desirable in films and coatings containing small particle size pigments as it reduces the tendency of such pigments to become unevenly distributed in the film or coating. This uneven distribution is sometimes referred to as flooding, floating or pigment migration.

In order to further illustrate the invention, various examples are set forth below. In these, as throughout the specification and claims, all parts and percentages are by weight and all temperatures in degrees Centigrade unless otherwise indicated.

### Example 1

Pyromellitic dianhydride (3.27 g) was dissolved in 45 g of acetone at room temperature in a round bottom flask equipped with a mechanical stirrer and vacuum distillation unit. Over a period of two hours, a solution containing 30 g of a primary amine-functionalized poly(oxypropylene (PO)/oxyethylene (EO)) methyl ether having an average equivalent molecular weight of about 2000 and a PO/EO ratio of 29/6, dissolved in 15 g of acetone was added and then the reaction mixture stirred for an additional hour. A charge of 1.53 g of 3-dimethylaminopropylamine was added to the reaction mixture over a period of 15 minutes and the reaction continued for another hour at which point the acetone was removed at 45° C. under vacuum. The resulting product (Dispersant 1) was a very thick light amber transparent polymer.

### Example 2

To a solution of 116 g of the dispersant of example 1 dissolved in 100 grams of acetone, 5.05 g of sulfuric acid (96.7%) was slowly added and stirred for 15 minutes. The acetone was then removed at room temperature under vacuum to yield a very thick light cream translucent HSO₄_{^{−}} salt of Dispersant 1 (Dispersant 2).

### Example 3

Pyromellitic dianhydride (3.27 g) was dissolved in 60 g of acetone at room temperature in a round bottom flask equipped with a mechanical stirrer and vacuum distillation unit to which 36 grams of a polyisobutylene amine (65%) having an average equivalent molecular weight of about 1500 was added over a period of one hour and the reaction mixture then stirred for an additional two hours. A charge of 1.53 grams of 3-dimethylaminopropylamine was added to the reaction mixture over 15 minutes and stirring was continued for an additional two hours. The acetone was removed at 45° C. under vacuum leaving a light amber translucent viscous liquid (Dispersant 3).

### Example 4

Pyromellitic dianhydride (3.25 g) was dissolved in 45 grams of acetone at room temperature. A solution of 30 grams of a primary amine-functionalized poly(PO/EO) methyl ether having an average equivalent molecular weight of about 2000 and a PO/EP ratio of 29/6, dissolved in 15 grams of acetone was added over two hours and the mixture then stirred for an additional hour. A charge of 30 grams of a primary amine-terminated hydrophilic poly(PO/EO) methyl ether having an average equivalent molecular weight of about 2000 and a PO/EO ratio of 10/31 was added over 20 minutes and then stirring was continued for an additional two hours at which time the acetone was removed under vacuum at 45° C. The resulting product was a light amber transparent polymer (Dispersant 4).

### Example 5

Pyromellitic dianhydride (3.27 g) was dissolved in 45 g of acetone at room temperature and then a solution of 30 g of a primary amine-functionalized poly(PO/EO) methyl ether having an average equivalent molecular weight of about 2000 and a PO/EO ratio of 29/6, dissolved in 15 g of acetone was added over two hours. The resulting mixture was stirred for an additional hour at which time 0.81 g of p-phenylene diamine was added and the stirring continued for an additional five hours. The acetone was removed under vacuum at 40° C. to yield an amber transparent polymer (Dispersant 5).

### Example 6

Pyromellitic dianhydride (13.09 g) was dissolved in 180 g of acetone at room temperature and then a solution of 120 g of a primary amine-terminated hydrophilic poly(PO/EO) methyl ether having an average equivalent molecular weight of about 2000 and a PO/EO ratio of 10/31, dissolved in 60 g of acetone was added over two hours. The resulting mixture was stirred for an additional hour at which time 6.9 g of poly(PO)diamine having an average equivalent molecular weight of about 115 was added and the stirring continued for an additional five hours. The acetone was removed under vacuum at 40° C. to yield a dark amber transparent polymer. This product was heated to 120° C. for 2.5 hrs to yield an transparent viscous liquid (Dispersant 6).

### Example 7

Pyromellitic dianhydride (3.27 g) was dissolved in 45 g of acetone at room temperature and then a solution of 30 g of a primary amine-terminated poly(PO/EO) methyl ether having an average equivalent molecular weight of about 2000 and a PO/EO ratio of 29/66, dissolved in 15 g of acetone was added over two hours. The resulting mixture was stirred for an additional hour at which time 2.04 g of N,N-dimethyl-1,4-phenylenediamine was added and the stirring continued for an additional five hours. The acetone was removed under vacuum at 40° C. to yield an amber transparent polymer (Dispersant 7).

### Example 8

A charge of 9.66 g of benzophenone-3,3′,4,4′-tetracarboxylic dianhydride was dissolved in 250 g of acetone at room temperature. A solution of 60 g of a primary amine-functionalized poly(PO/EO) methyl ether having an average equivalent molecular weight of about 2000 and a PO/EO ratio of 29/6, dissolved in 30 g of acetone, was added over two hours and then stirred for an additional hour. A charge of 3.1 g of 3-dimethylpropylamine was added over 15 minutes and the stirring continued for an additional four hours at which time the acetone was removed at room temperature. The product was a very thick light amber translucent polymer (Dispersant 8).

### Example 9

Solvent borne dispersions were prepared by mixing pigment (DPP Red BO (CIBA), Red 202 (Sun Chemical), Red 179 (Sun Chemical), Blue 15:1 (Sun Chemical), Blue 15:2 (Sun Chemical), carbon black 250 (Degussa)), with oligomer CN2102E and monomer SR306 (Sartomer), and n-Butyl Propionate (Aldrich), as set forth below, with or without dispersant, on Speed Mixer DAC 150FVZ (Hauschild) for 4 minutes with an equal weight (unless otherwise indicated) of sand as grinding medium. The viscosities of the resulting dispersions were measured by flow plate technique.

| | % | Grams | Flow, cm/sec |
|---|---|---|---|
| Formula 1: | | | |
| DPP Red BO | 56.0 | 2. | 22 cm/6 sec |
| Dispersant 1 | 5.0 | 0.25 | |
| n-Butyl Propionate | 39.0 | 1.95 | |
| Control 1: | | | |
| DPP Red BO | 56.0 | 2.8 | No flow |
| n-Butyl Propionate | 44.0 | 2.2 | |
| Formula 2: | | | |
| Red 202 | 40.0 | 2.0 | 8 cm/30 sec |
| Dispersant 1 | 5.0 | 0.25 | |
| n-Butyl Propionate | 55.0 | 2.75 | |
| Control 2: | | | |
| Red 202 | 40.0 | 2.0 | No flow |
| n-Butyl Propionate | 60.0 | 3.0 | |
| Formula 3: | | | |
| Red 179 | 49.0 | 2.45 | 18 cm/10 sec |
| Dispersant 1 | 5.0 | 0.25 | |
| n-Butyl Propionate | 46.0 | 2.3 | |
| Control 3: | | | |
| Red 179 | 49.0 | 2.45 | No flow |
| n-Butyl Propionate | 51.0 | 2.55 | |
| Formula 6: | | | |
| Red 202 | 27.3 | 1.5 | 21 cm/24 sec |
| Dispersant 4 | 9.1 | 0.5 | |
| n-Butyl Propionate | 63.6 | 3.5 | |
| Control 6: | | | |
| Red 202 | 27.3 | 1.5 | No flow |
| n-Butyl Propionate | 72.7 | 4.0 | |
| Formula 9: | | | |
| Blue 15:2 | 40.2 | 2.35 | 20.5 cm/38 sec |
| Dispersant 7 | 4.3 | 0.25 | |
| n-Butyl Propionate | 55.5 | 3.25 | |
| Control 9: | | | |
| Blue 15:2 | 40.2 | 2.35 | No flow |
| n-Butyl Propionate | 59.8 | 3.5 | |
| Formula 10: | | | |
| Blue 15:1 | 30.0 | 1.5 | 22 cm/8 sec |
| Dispersant 8 | 5.0 | 0.25 | |
| n-Butyl Propionate | 65.0 | 3.25 | |
| Control 10: | | | |
| Blue 15:1 | 30 | 1.5 | No flow |
| n-Butyl Propionate | 70 | 3.5 | |
| Formula 11: | | | |
| Blue 15:2 | 30.0 | 1.5 | 15 cm/8 sec |
| Dispersant 8 | 5.0 | 0.25 | |
| n-Butyl Propionate | 65.0 | 3.25 | |
| Control 11: | | | |
| Blue 15:2 | 30 | 1.5 | No flow |
| n-Butyl Propionate | 70 | 3.5 | |
| Formula 14: | | | |
| Red 202 | 35.0 | 1.75 | 22 cm/6 sec |
| Dispersant 9 | 6.0 | 0.3 | |
| n-Butyl Propionate | 59.0 | 2.95 | |
| Control 14: | | | |
| Red 202 | 35.0 | 1.75 | No flow |
| n-Butyl Propionate | 65.0 | 3.25 | |
| Formula 16: | | | |
| Blue 15:2 | 40.0 | 2.2 | 9.5 cm/47 sec |
| Dispersant 10 | 4.0 | 0.22 | |
| n-Butyl Propionate | 56.0 | 3.08 | |
| Control 16: | | | |
| Blue 15:2 | 40.0 | 2.0 | No flow |
| n-Butyl Propionate | 60 | 3.0 | |
| Formula 17: | | | |
| Red 202 | 35.0 | 1.75 | 21 cm/24 sec |
| Dispersant 11 | 6.0 | 0.3 | |
| n-Butyl Propionate | 59.0 | 2.95 | |
| Control 17 | | | |
| Red 202 | 35.0 | 1.75 | No flow |
| n-Butyl Propionate | 65.0 | 3.25 | |
| Formula 18: | | | |
| Carbon Black 250 | 55.1 | 4.3 | 20 cm/37 sec |
| Dispersant 11 | 3.2 | 0.25 | |
| n-Butyl Propionate | 41.7 | 3.25 | |
| Control 18: | | | |
| Carbon Black 250 | 55.1 | 4.3 | No flow |
| n-Butyl Propionate | 44.9 | 3.5 | |
| Formula 19: | | | |
| Red 202 | 35.0 | 1.75 | 3.5 cm/35 sec |
| Dispersant 12 | 6.0 | 0.3 | |
| n-Butyl Propionate | 59.0 | 2.95 | |
| Control 19: | | | |
| Red 202 | 35.0 | 1.75 | No flow |
| n-Butyl Propionate | 65.0 | 3.25 | |
| Formula 20: | | | |
| Carbon Black 250 | 55.1 | 4.3 | 15 cm/37 sec |
| Dispersant 12 | 3.2 | 0.25 | |
| n-Butyl Propionate | 41.7 | 3.25 | |
| Control 20: | | | |
| Carbon Black 250 | 55.1 | 4.3 | No flow |
| n-Butyl Propionate | 44.9 | 3.5 | |

### Example 10

The UV Flexo dispersions set forth below were prepared by mixing carbon black 250 (Degussa), oligomer CN2102E and monomer SR306 (Sartomer), with or without dispersant, on a Speed Mixer DAC 150FVZ (from Hauschild) for 5 minutes with an equal weight of sand as grinding medium. The viscosities of the resulting dispersions were measured by flow plate technique.

| | % | Amount, g | Flow, cm/sec |
|---|---|---|---|
| Formula 4: | | | |
| Carbon Black 250 | 30.0 | 1.5 | 21.5 cm/30 sec |
| Dispersant 2 | 6.0 | 0.3 | |
| Oligomer CN2102E | 32.0 | 1.6 | |
| Monomer SR306 | 32.0 | 1.6 | |
| Control 4: | | | |
| Carbon Black 250 | 30.0 | 1.5 | No flow |
| Oligomer CN2102E | 35.0 | 1.75 | |
| Monomer SR306 | 35.0 | 1.75 | |
| Formula 7: | | | |
| Carbon Black 250 | 34.0 | 1.8 | 20 cm/26 sec |
| Dispersant 5 | 5.6 | 0.3 | |
| Oligomer CN2102E | 30.2 | 1.6 | |
| Monomer SR306 | 30.2 | 1.6 | |
| Control 7: | | | |
| Carbon Black 250 | 34.0 | 1.8 | No flow |
| Oligomer CN2102E | 33.0 | 1.75 | |
| Monomer SR306 | 33.0 | 1.75 | |
| Formula 12: | | | |
| Carbon Black 250 | 30.0 | 1.5 | 18 cm/40 sec |
| Dispersant 8 | 6.0 | 0.3 | |
| Oligomer CN2102E | 32.0 | 1.6 | |
| Monomer SR306 | 32.0 | 1.6 | |
| Control 12: | | | |
| Carbon Black 250 | 30.0 | 1.5 | No flow |
| Oligomer CN2102E | 35.0 | 1.75 | |
| Monomer SR306 | 35.0 | 1.75 | |
| Formula 21: | | | |
| Carbon Black 250 | 30.0 | 1.5 | 21 cm/27 sec |
| Dispersant 12 | 6.0 | 0.3 | |
| Oligomer CN2102E | 32.0 | 1.6 | |
| Monomer SR306 | 32.0 | 1.6 | |
| Control 21: | | | |
| Carbon Black 250 | 30.0 | 1.5 | No flow |
| Oligomer CN2102E | 35.0 | 1.75 | |
| Monomer SR306 | 35.0 | 1.75 | |
| Formula 22: | | | |
| Carbon Black 250 | 30.0 | 1.5 | 16.5 cm/27 sec |
| Dispersant 13 | 6.0 | 0.3 | |
| Oligomer CN2102E | 32.0 | 1.6 | |
| Monomer SR306 | 32.0 | 1.6 | |
| Control 22: | | | |
| Carbon Black 250 | 30.0 | 1.5 | No flow |
| Oligomer CN2102E | 35.0 | 1.75 | |
| Monomer SR306 | 35.0 | 1.75 | |

### Example 11

The oil based dispersions set forth below were prepared by mixing carbon black 250 (Degussa), and Magiesol 47 oil (Magie Bros.), with or without dispersant, on a Speed Mixer DAC 150FVZ (from Hauschild) for 4 minutes with an equal weight of sand as grinding medium. The viscosities of the resulting dispersions were measured by flow plate technique.

| | % | Amount, g | Flow, cm/sec |
|---|---|---|---|
| Formula 5: | | | |
| Carbon Black 250 | 50.0 | 2.5 | 20.5 cm/90 sec |
| Dispersant 3 (68%) | 7.2 | 0.36 | |
| 47 oil | 42.8 | 2.14 | |
| Control 5: | | | |
| Pigment | 50.0 | 2.5 | No flow |
| 47 oil | 50.0 | 2.5 | |

### Example 12

The water borne dispersions set forth below were prepared by mixing carbon black 250 (Degussa) or Red 202 (Sun Chemical), with or without dispersant, for 30 minutes with sand as grinding medium. The viscosities of the resulting dispersions were measured by Brookfield DV-I+ Viscometer (with spindle #4 at 50 rpm).

| | % | Amount, g | Viscosity |
|---|---|---|---|
| Formula 8: | | | |
| Carbon Black 250 | 48.0 | 12.48 | 264 cps |
| Dispersant 6 | 5.2 | 1.35 | |
| Water | 46.8 | 12.17 | |
| Control 8: | | | |
| Carbon Black 250 | 48.0 | 6.24 | Very thick |
| Water | 52.0 | 6.76 | |
| Formula 23: | | | |
| Carbon Black 250 | 48.0 | 12.48 | 360 cps |
| Dispersant 14 | 5.2 | 1.35 | |
| Water | 46.8 | 12.17 | |
| Control 23: | | | |
| Carbon Black 250 | 48.0 | 6.24 | Very thick |
| Water | 52.0 | 6.76 | |
| Formula 24: | | | |
| Red 202 | 30.0 | 12.0 | 372 cps |
| Dispersant 15 | 10.0 | 4.0 | |
| Water | 60.0 | 24.0 | |
| Control 24: | | | |
| Red 202 | 30.0 | 12.0 | Very thick |
| Water | 70.0 | 28.0 | |

### Examples 13

Pyromellitic dianhydride (2.18 g) was dissolved in 60 g of 2-butanone at room temperature in a round bottom flask equipped with a mechanical stirrer and vacuum distillation unit. Over a period of two hours a solution containing 20.4 g of a primary amino-functionalized poly(PO/EO) methyl ether having an average equivalent molecular weight of about 2000 and PO/EO ratio equal 29/6, dissolved in 5 g of 2-butanone was added and then the reaction mixture was stirred for an additional hour. A charge of 1.36 g of 2[2-(dimethylamino)ethoxy]ethanol was added to the reaction mixture over a period of 15 minutes and the reaction continued for another hour at which point 2-butanone was removed at 50° C. under vacuum. The resulting product (Dispersant 9) was a very thick light amber transparent polymer.

### Example 14

Pyromellitic dianhydride (16.36 g) was dissolved in 195 g of acetone at room temperature in a round bottom flask equipped with a mechanical stirrer and vacuum distillation unit. Over a period of three hours, a solution containing 151.5 g of a primary amine-functionalized poly(PO/EO) methyl ether having an average equivalent molecular weight of about 2000 and PO/EO ratio equal 29/6, dissolved in 30 g of acetone was added and the reaction mixture was stirred for an additional hour. A charge of 11.17 g of 2-(dimethylamino)ethanethiol hydrochloride was added to the reaction mixture and the reaction continued for 45 hours at which point the acetone was removed at 40° C. under vacuum. The resulting product (Dispersant 10) was a very thick white polymer.

### Example 15

Pyromellitic dianhydride (21.8 g) was dissolved in 260 g of acetone at room temperature and then a solution of 202 g of a primary amine-terminated poly(PO/EO) methyl ether having an average equivalent molecular weight of about 2000 and PO/EO ratio equal 29/6, dissolved in 40 g of acetone was added over three hours. The resulting mixture was stirred for an additional hour at which time 13.05 g of 1-(2-aminoethyl)piperazine was added and the stirring continued for an additional five hours. The acetone was removed under vacuum at 40° C. to yield a light amber transparent polymer (Dispersant 11).

### Example 16

Pyromellitic dianhydride (2.18 g) was dissolved in 26 g of acetone at room temperature and then a solution of 20.2 g of a primary amine-terminated poly(PO/EO) methyl ether having an average equivalent molecular weight of about 2000 and PO/EO ratio equal 29/6, dissolved in 4 g of acetone was added over two hours. The resulting mixture was stirred for an additional hour at which time 0.65 g of 1-(2-aminoethyl)piperazine was added and the stirring continued for an additional five hours. The acetone was removed under vacuum at 40° C. to yield a light amber transparent polymer (Dispersant 12). Then the polymer was heated to 120° C. for 2.5 hrs to yield a dark amber transparent viscous liquid (Dispersant 13).

### Example 17

Pyromellitic dianhydride (21.8 g) was dissolved in 260 g of acetone at room temperature and then a solution of 202 g of a primary amine-terminated hydrophilic poly(PO/EO) methyl ether having an average equivalent molecular weight of about 2000 and PO/EO ratio equal 10/31, dissolved in 40 g of acetone was added over two hours. The resulting mixture was stirred for an additional hour at which time 13.05 g of 1-(2-aminoethyl)piperazine was added and the stirring continued for an additional five hours at which time 18.0 g of Epon 826 (Bisphenol A diglycidyl ether from Hexion with epoxy equivalent weight 180) and the stirring continued for an additional 1 hour. The acetone was removed under vacuum at 35° C. to yield a light amber transparent polymer. Then the polymer was heated to 130° C. for 2 hrs to yield a dark amber transparent viscous liquid (Dispersant 14).

### Example 18

To 69.6 g of the dispersant of example 1, 6.1 g of triethylamine (99.5%) was slowly added and stirred for 60 minutes at room temperature to yield a very thick light yellow translucent salt of Dispersant 1 (Dispersant 15).

Various changes and modifications can be made in the products and processes described above with departing from the spirit and scope of the invention. The various embodiments which have been set forth were for the purpose of illustration only and are not intended to limit the invention.

1-20. (canceled)

21. A method of preparing a dispersant which comprises reacting at least one dianhydride with at least two reactants which are different from each other in a substantially anhydrous solvent, wherein each reactant comprises at least one functional group independently selected from the group consisting of amine, hydroxyl and thiol, and at least one of which reactants is polymeric.

22. A method of dispersing a pigment which comprises combining a liquid or plastic material, pigment and the dispersant of claim 21.

23. A process which comprises heating a pigmented composition containing the dispersant of claim 21 about 70° C.

24-25. (canceled)

26. A process of coloring a surface of a substrate which comprises applying the combination of the dispersant of claim 21 and a pigment on the surface.

27. The method of claim 21, wherein the dianhydride is of the formula: 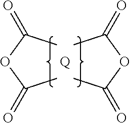
wherein Q is a carbon-containing linking group.

28. The method of claim 27, Q is aromatic, and at least one reactant is an amine-terminated polymer.

29. The method of claim 28, wherein all reactants have at least one primary or secondary amino group.

30. The method of claim 28, wherein at least one reactant has at least one hydroxyl group.

31. The method of claim 28, wherein at least one reactant has at least one thiol group.

32. The method of claim 21, wherein the dispersant is a material comprising a unit of the structure 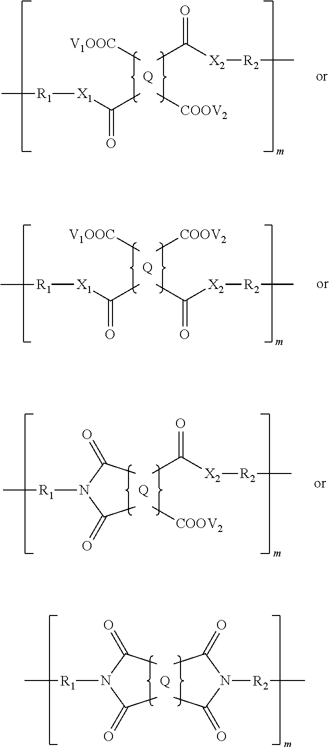
or combination thereof, wherein Q is a carbon-containing linking group; each X₁ and X₂ are independently NZ, O or S; each R₁, R₂ and Z are independently hydrogen or a linear, branched or cyclic aliphatic or aromatic carbon-containing group or a combination thereof, each V₁ and V₂ independently are hydrogen or the residue of an entity reactive with a —CO₂H group, provided that at least one is polymeric; and m is an integer from 1 to 10.

33. The method of claim 32, wherein m is more than 1 and at least two C═O moieties of the dianhydrides are linked by a symmetric or asymmetric difunctional reactant having a primary or secondary amino, hydroxyl or thiol functional group, or a combination thereof.

34. The method of claim 32, wherein Q is arylene, and X₁ and X₂ are NZ.

35. The method of claim 32, wherein Q is arylene, and one of X₁ and X₂ is NZ and the other is S or O.

36. The method of claim 32, wherein at least one of R₁ or R₂ is a member selected from the group consisting of poly(oxyalkylene) and polyalkylene.
